(19) United States
(12) Patent Application Publication
(10) Pub. No.: US 2017/0095413 A1
(43) Pub. Date: Apr. 6, 2017

(54) COSMETIC PROCESS FOR ATTENUATING WRINKLES

(71) Applicant: L'OREAL, Paris (FR)

(72) Inventors: Xavier SCHULTZE, Les Pavillons Sous Bois (FR); Bertrand LION, Paris (FR); Andrew GREAVES, Magny-Le-Hongre (FR); Anne POTTER, Neuilly-Sur-Seine (FR)

(21) Appl. No.: 15/311,648

(22) PCT Filed: May 27, 2015
(86) PCT No.: PCT/EP2015/061661

§ 371 Date: Nov. 16, 2016

(30) Foreign Application Priority Data
May 28, 2014 (FR) 1454825

(51) Int. Cl. A61K 8/73; A61K 8/41; A61K 8/04; A61Q 19/08

(52) U.S. Cl. CPC A61K 8/735; A61Q 19/08; A61K 8/41; A61K 8/042; A61K 2800/54

(57) ABSTRACT

The invention relates to a cosmetic process for caring for the skin, more particularly facial skin, in particular wrinkled skin, comprising:
either the topical application to the skin of an extemporaneous mixture of a cosmetic composition comprising a hyaluronic acid polymer grafted with (meth)acrylate groups and an amine compound;
or the sequential application to the skin of a cosmetic composition comprising a hyaluronic acid polymer grafted with (meth)acrylate groups and of an amine compound having one or more primary amine and/or secondary amine groups.

The invention also relates to the use of said grafted hyaluronic acid polymer and of said amine compound as a skin tensioning agent.

The invention also relates to a cosmetic composition obtained by mixing a cosmetic composition comprising said grafted hyaluronic acid polymer and said amine compound.

The invention relates to a kit comprising a first cosmetic composition comprising said grafted hyaluronic acid polymer and a second composition comprising said amine compound, the first and second compositions each been packaged in a distinct packaging assembly.

The present invention relates to a skincare process, in particular a cosmetic skincare process, intended to attenuate wrinkles, comprising the application to the skin of the mixture of a composition comprising a grafted hyaluronic acid polymer and of a particular amine compound, and to the use of this mixture as a skin tensioning agent.

During the ageing process, various signs appear on the skin, which are very characteristic of this ageing, resulting in particular in a modification of skin structure and functions. The main clinical signs of skin ageing are in particular the appearance of fine lines and deep wrinkles, which increase with age.

It is known practice to treat these signs of ageing using cosmetic or dermatological compositions containing active agents capable of combating ageing, such as a-hydroxy acids, β-hydroxyacids and retinoids. These active agents act on wrinkles by eliminating dead skin cells and by accelerating the cell renewal process. However, these active agents have the drawback of only being effective for the treatment of wrinkles after a certain application time. However, it is increasingly sought to obtain a quick or immediate effect of the active agents used, rapidly resulting in smoothing out of wrinkles and fine lines and in the disappearance of the signs of fatigue.

Hyaluronic acid is known for its skin tensioning properties.

The inventors have discovered that a hyaluronic acid polymer grafted with (meth)acrylate groups combined with a particular amine compound has an improved tensioning effect on the skin and thus makes it possible to attenuate wrinkles on the skin, notably quickly or immediately after application on the skin. Furthermore, the tensioning effect obtained also exhibits good water resistance, and therefore good persistence with respect to water.

Polymers of hyaluronic acid grafted with (meth)acrylate groups are described in document WO 2007/106738 and the publications J. Burdick et al “Controlled degradation and mechanical behavior photopolymerized hyaluronic acid networks”, Biomacromolecules, 2005, 6, pages 386-391; Mark Grinstaff “Photocrosslinkable polysaccharides for in situ hydrogel formation”, Journal of biomedical materials research, 2001, volume 55, Issue 2, pages 115-121. They have been used to form hydrogels after crosslinking.

More specifically, a subject of the present invention is a process, in particular a cosmetic process, for caring for the skin, more particularly facial skin, in particular wrinkled skin, comprising: either the topical application to the skin of an extemporaneous mixture of a cosmetic composition comprising a hyaluronic acid polymer grafted with (meth)acrylate groups and an amine compound having one or more primary amine and/or secondary amine groups;

or the sequential application to the skin of a cosmetic composition comprising a hyaluronic acid polymer grafted with (meth)acrylate groups and of an amine compound having one or more primary amine and/or secondary amine groups, or of a cosmetic composition containing same.

The process according to the invention is in particular intended for smoothing out human facial and/or body skin and/or for decreasing or effacing the signs of skin ageing, in particular for reducing or effacing wrinkles and/or fine lines on the skin.

A subject of the invention is also the use, in particular cosmetic use, as a skin, in particular wrinkled skin, tensioning agent, of a grafted hyaluronic acid polymer as previously defined, or of a cosmetic composition composition containing same, mixed with the amine compound as previously defined.

A subject of the invention is also a composition, in particular a cosmetic composition, obtained by mixing a cosmetic composition comprising a grafted polymer as previously defined and an amine compound as previously defined or a composition, in particular a cosmetic composition, containing same.

A subject of the invention is also a kit comprising a first composition, in particular a cosmetic composition, comprising a grafted polymer as previously defined and a second composition comprising an amine compound as previously described, or a composition, in particular a cosmetic composition, comprising same, the first and second compositions each being packaged in a distinct packaging assembly.

The composition packaging assembly is, in a known manner, any packaging that is suitable for storing cosmetic compositions (especially a bottle, tube, spray bottle or aerosol bottle).

Such a kit allows the skin treatment process according to the invention to be performed.

The term “tensioning agent” is intended to mean compounds capable of having a noticeable tensioning effect, i.e. of smoothing out the skin and immediately reducing, or even causing to disappear, the wrinkles and fine lines.

The tensioning effect may be characterized by means of an in vitro retraction test as described in Example 5 hereinafter.

Hyaluronic acid is a linear glycosaminoglycan composed of repeating D-glucuronic acid and N-acetyl-D-glucosamine units linked together via alternating beta-1,4 and beta-1,3 glycosidic linkages.

Preferably, the grafted hyaluronic acid polymer has a weight-average molecular weight ranging from 5000 to 1 000 000 daltons, more preferentially ranging from 10 000 to 500 000 daltons, and even more preferentially ranging from 15 000 to 350 000 daltons.

The molecular weight can be determined in particular by liquid chromatography, the eluent being 0.1 M sodium chloride and 330 mg/l of sodium azide in water, with dextran as standard, and Wyatt Optilab T-Rex refractometer and Wyatt Dawn-Heleos II light scattering detectors.

Advantageously, the grafted hyaluronic acid polymer has a degree of grafting with (meth)acrylate groups ranging from 10% to 80% or 20% to 80%, preferably ranging from 40% to 70%, and preferentially ranging from 45% to 65%. The degree of grafting corresponds to the mole percentage of hydroxyl groups of the hyaluronic acid which are grafted with a (meth)acrylate group.

By way of example, a degree of grafting of 50% corresponds to 2 acrylate groups grafted onto the 4 hydroxyls of the repeating unit of the hyaluronic acid.

The grafting of hyaluronic acid with (meth)acrylate groups results from the presence of a (meth)acrylate ester group formed with the free hydroxyls of hyaluronic acid.

Preferably, the hyaluronic acid is grafted with acrylate groups.

The hyaluronic acid grafted with (meth)acrylate groups can be obtained by reaction of the hyaluronic acid with (meth)acrylic anhydride. The reaction is advantageously carried out in a basic aqueous medium, in particular in the presence of an organic or inorganic base such as, for example, sodium hydroxide. Preferably, the reaction is carried out at a temperature ranging from 5 to 10° C., in particular for a period of time ranging from 24 hours to 48 hours.

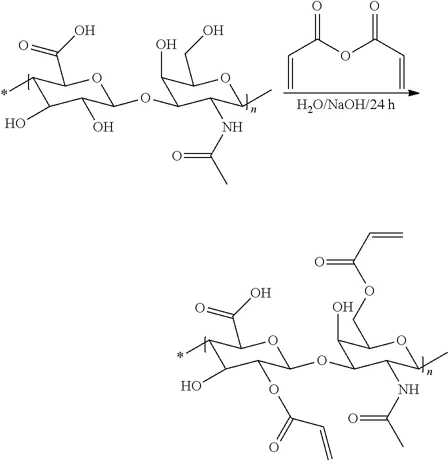

Various degrees of grafting with the (meth)acrylate groups can be obtained by varying the amount of (meth)acrylic anhydride used proportionally to the amount of hyaluronic acid.

The grafted hyaluronic acid polymer as previously defined may be present in the composition used according to the invention in a content ranging from 0.1% to 10% by weight, relative to the total weight of the composition, preferably ranging from 0.5% to 10% by weight of active material, preferentially ranging from 1% to 8% by weight, and more preferentially ranging from 1% to 6% by weight.

The amine compound used in the process according to the invention is chosen from amine compounds having one or more primary amine and/or secondary amine groups. It can thus be chosen from monoamine, diamine, triamine or polyamine compounds. According to a first embodiment of the invention, the amine compound is a compound comprising from 2 to 20 carbon atoms, and in particular a non-polymeric compound. The term “non-polymeric” compound is intended to mean a compound which is not directly obtained by means of a monomer polymerization reaction

As amine compounds, mention may be made of n-butylamine, tert-butylamine, isobutylamine, propylamine, n-hexylamine, glycine, ethanolamine, 3-aminopropanol, dopamine, 7-amino-4-methylcoumarin, 1,4-bis(3-aminopropyl)piperazine, 3-aminopropyltriethoxysilane (APTES), 3-aminophenylboronic acid, N-méthyl-1,3-diaminopropane, N-propyl-1,3-diaminopropane, N-isopropyl-1,3-diaminopropane, N-cyclohexyl-1,3-diaminopropane, 2-(3-aminopropylamino)ethanol, 3-(2-aminoethyl)aminopropylamine, bis(3-aminopropyl)amine, méthylbis(3-aminopropyl)amine, N-(3-aminopropyl)-1,4-diaminobutane, N,N-dimethyldipropylenetriamine, 1,2-bis(3-aminopropylamino)ethane, N,N′-bis(3-aminopropyl)-1,3-propanediamine, ethylenediamine, 1,3-propylenediamine, 1,4-butylenediamine, lysine, cystamine, xylènediamine, tris(2-aminoethyl)amine and spermidine.

Preferably, the amine compound is chosen from n-butylamine, 3-aminopropanol, dopamine, 7-amino-4-methylcoumarin, 1,4-bis(3-aminopropyl)piperazine, 3-aminopropyltriethoxysilane (APTES), 3-aminophenylboronic acid, N-méthyl-1,3-diaminopropane, N-propyl-1,3-diaminopropane, N-isopropyl-1,3-diaminopropane, N-cyclohexyl-1,3-diaminopropane, 2-(3-aminopropylamino)ethanol, 3-(2-aminoethyl)aminopropylamine, bis(3-aminopropyl)amine, méthylbis(3-aminopropyl)amine, N-(3-aminopropyI)-1,4-diaminobutane, N,N-dimethyldipropylenetriamine, 1,2-bis(3-aminopropylamino)ethane, N,N′-bis(3-aminopropyl)-1,3-propanediamine, ethylenediamine and lysine.

The amine compound may also be chosen from amine-comprising polymers, in particular having a weight-average molecular weight ranging from 500 to 1 000 000, preferably ranging from 500 to 500 000, and preferentially ranging from 500 to 100 000.

As amine-comprising polymer, use may be made of poly((C₂-C₅)alkyleneimines), and in particular polyethyleneimines and polypropyleneimines, especially poly(ethyleneimine)s (for example the product sold under the reference 46,852-3 by the company Aldrich Chemical); poly(allylamine) (for example the product sold under the reference 47,913-6 by the company Aldrich Chemical); polyvinylamines and copolymers thereof, in particular with vinylamides; mention may in particular be made of vinylamine/vinylformamide copolymers such as those sold under the name Lupamin® 9030 by the company BASF; polyamino acids which have NH₂ groups, such as polylysine, for example the product sold by the company JNC Corporation (formally Chisso); aminodextran, such as the product sold by the company CarboMer Inc; amino polyvinyl alcohol, such as the product sold by the company CarboMer Inc, acrylamidopropylamine-based copolymers; chitosans;

polydimethylsiloxanes comprising primary amine groups at the chain end or on side chains, for example aminopropyl end or side groups, for instance those of formula (A) or (B) or (C):

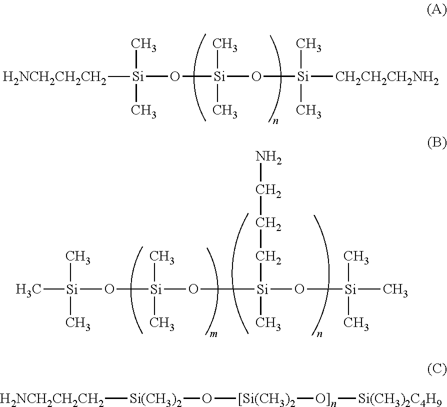

In formula (A): the value of n is such that the weight-average molecular weight of the silicone is between 500 and 55 000. As an example of aminosilicone (A), mention may be made of those sold under the names DMS-A11, DMS-A12, DMS-A15, DMS-A21, DMS-A31, DMS-A32 and DMS-A35 by the company Gelest.

In formula (B), the values of n and m are such that the weight-average molecular weight of the silicone is between 1000 and 55 000. As examples of silicone (B), mention may be made of those sold under the names AMS-132, AMS-152, AMS-162, AMS-163, AMS-191 and AMS-1203 by the company Gelest.

In formula (C), the value of n is such that the weight-average molecular weight of the silicone is between 500 and 3000. As an example of silicone (C), mention may be made of those sold under the names MCR-A11 and MCR-A12 by the company Gelest;

amodimethicones of formula (D):

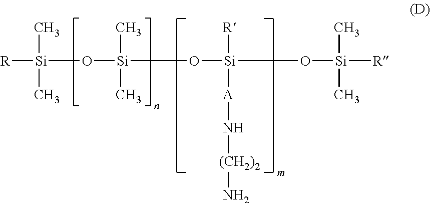

in which R, R′ and R″, which may be identical or different, each represent a C₁-C₄ alkyl or hydroxyl group, A represents a C₃ alkylene group and m and n are such that the weight-average molecular mass of the compound is between 5000 and 500 000 approximately;

polyether amines, in particular known under the reference Jeffamine from the company Huntsman; and in particular:

polyethylene glycols and/or polypropylene glycols comprising an amine function at the chain end (monoamine or diamine), such as those sold under the names Jeffanine M-600, M-1000, M-2005, M-2070, D-230, D-400, D-2000, D-4000, ED-600, ED-9000 and ED-2003;

polytetrahydrofurans (or polytetramethylene glycols) comprising an amine function at the chain end (monoamine or diamine), polybutadienes comprising an amine function at the chain end (monoamine or diamine);

dendrimers and polymers which are hyperbranched, comprising a primary or secondary amine function (PANAM);

poly(meth)acrylates or poly(meth)acrylamides bearing primary or secondary amine side functions, such as poly(3-aminopropyl)méthacrylamide or poly(2-aminoethyl) méthacrylate.

As amine-comprising polymer, use is preferably made of polyethyleneimine, polylysine, chitosans and polyethylene oxides and/or polypropylene oxides comprising amine end groups.

Preferentially, the amine compounds used in the process according to the invention are chosen from ethylenediamine, lysine and 3-am inopropyltriethoxysilane (APTES). More preferentially, ethylenediamine is used.

Advantageously, the amine compound used in the process according to the invention is used according to an amine compound/grafted hyaluronic acid (meth)acrylate group mole ratio ranging from 0.1 to 10, preferably ranging from 0.1 to 5, and preferentially ranging from 0.1 to 2.

The amine compound in contact with the grafted hyaluronic acid reacts with the ethylenic unsaturations of the (meth)acrylate groups grafted onto the hyaluronic acid so as to form amine bonds with the end carbon of the ethylenic unsaturation of the (meth)acrylate group (this reaction is known as the Michael reaction).

Examples of this reaction are illustrated in the following schemes:

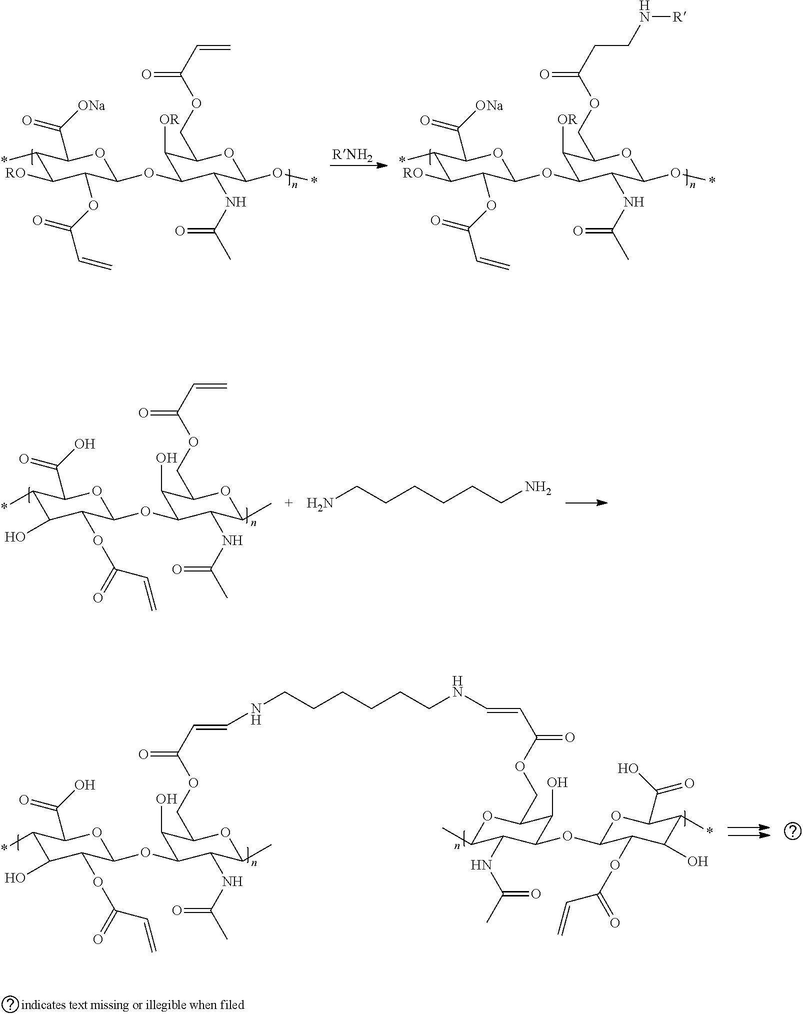

When the grafted hyaluronic acid comprises methacrylate groups, a catalyst can be used in the presence of the amine compound. This catalyst makes it possible to obtain good reactivity of the amine compound on the ethylenic unsaturation of the methacrylate group.

The catalyst can be chosen from the catalysts described in the articles Tetrahedron Letters 48 (2007) pages 141-143 and Tetrahedron Letters 46 (2005) pages 8329-8331, and also in the articles cited in these two articles.

As an example of a catalyst, mention may be made of Lewis acids, such as boric acid, aluminium chloride or cerium chloride, and also phosphines, such as trimethylphosphine (trialkylphosphine), phenyldimethylphosphine (dialkylarylphosphine), diphenylmethylphosphine (alkyldiarylphosphine), triphenylphosphine (triarylphosphine), tricarboxyethylphosphine, and the oxide equivalents.

The composition used according to the invention is generally suitable for topical application to the skin and thus generally comprises a physiologically acceptable medium, i.e. a medium that is compatible with the skin and/or skin appendages. It is preferably a cosmetically acceptable medium, i.e. a medium which has a pleasant colour, odour and feel and which does not cause any unacceptable discomfort (stinging, tautness or redness) liable to discourage the consumer from using this composition.

The composition used according to the invention may be in any galenic form conventionally used for a topical application and especially in the form of dispersions of aqueous gel or lotion type, emulsions of liquid or semi-liquid consistency of the milk type, obtained by dispersing a fatty phase in an aqueous phase (O/W) or vice versa (W/O), or suspensions or emulsions of soft, semi-solid or solid consistency of the cream or gel type, or alternatively multiple emulsions (W/O/W or O/W/O), microemulsions, vesicular dispersions of ionic and/or nonionic type, or wax/aqueous phase dispersions. These compositions are prepared according to the usual methods.

According to one preferred embodiment of the invention, the composition is in the form of an O/W emulsion or an aqueous gel.

Advantageously, the composition used according to the invention comprises water, in particular in a content which can range from 10% to 99% by weight and preferably ranging from 50% to 99% by weight, relative to the total weight of the composition.

The composition used according to the invention may also contain one or more attachments commonly used in the cosmetics field, such as emulsifiers, preservatives, sequestering agents, fragrances, thickeners, oils, waxes or film-forming polymers.

Needless to say, those skilled in the art will take care to select this or these optional additional compound(s), and/or the amount thereof, such that the anti-wrinkle properties of the composition according to the invention are not, or are not substantially, adversely affected by the envisaged addition.

According to a first embodiment of the process according to the invention, an extemporaneous mixture of a cosmetic composition comprising the (meth)acrylate-grafted hyaluronic acid polymer and of an amine compound, or a cosmetic composition containing same, as previously described, is applied to the skin. The extemporaneous mixture is advantageously prepared less than 5 minutes before it is applied to the skin, and preferably less than 3 minutes.

According to a second embodiment of the process according to the invention, the cosmetic composition comprising the (meth)acrylate-grafted hyaluronic acid polymer is first applied to the skin, then the amine compound or a cosmetic composition containing same is applied. The application of the amine compound can be carried out after a time of between 5 minutes and one hour after having applied the grafted hyaluronic acid polymer.

According to a third embodiment of the process according to the invention, the amine compound, or a cosmetic composition containing same, is first applied to the skin, then the cosmetic composition comprising the (meth)acrylate-grafted hyaluronic acid polymer is applied. The application of the grafted hyaluronic acid polymer can be carried out after a time of between 5 minutes and one hour after having applied the amine compound.

The application of the cosmetic composition used according to the invention is carried out according to the usual techniques, for example by application (in particular of creams, gels, sera or lotions) to the skin intended to be treated, in particular facial and/or neck skin, especially the skin of the area around the eyes. In the context of this process, the composition may, for example, be a care composition.

The invention will now be described with reference to the examples that follow, which are given as non-limiting illustrations.

### EXAMPLE 1

### Hyaluronic Acid 60% Functionalized with Acrylic Anhydride

In a thermostated reactor, 5 g of hyaluronic acid (Hyacare® 50 from Evonik) were dissolved in 100 ml of water and the mixture was maintained at a temperature of 7° C., then 14.8 g of acrylic anhydride were added dropwise over the course of approximately 2 min. The pH was adjusted to 7.7 by slowly adding (over the course of approximately one hour) sodium hydroxide at 30% in water (7 M). The mixture was left to react for 24 hours.

The mixture obtained was purified by dialysis (polymer in 150 ml of water, 3.3% by weight) on a Spectra/Por® 15 kDa membrane for 5 days in 5 litres of water (water changed 4 times, i.e. 20 litres in total), then the mixture of the purified fraction was lyophilized by freezing with a bath of dry ice+acetone at −80° C., then by placing the frozen mixture in a lyophilization apparatus for 4 days. 2.5 g of a white solid were obtained.

Analyses:

¹H NMR D₂O: 2.45 (7.36/3) OH units functionalized for 4 OH units available.

The hyaluronic acid obtained is 60%-functionalized with acrylate groups.

### EXAMPLE 2

### Hyaluronic Acid 50% Functionalized with Acrylic Anhydride

The polymer was prepared according to the procedure described in Example 1, using 5 g of hyaluronic acid (Hyacare® 50 from Evonik) and 7.9 g of acrylic anhydride.

After purification, 2.5 g of a white solid were obtained.

Analyses:

¹H NMR D₂O: 1.96 OH units functionalized for 4 OH units available.

The hyaluronic acid obtained is 50%-functionalized with acrylate groups.

### EXAMPLE 3

### Hyaluronic Acid 18% Functionalized with Acrylic Anhydride

The polymer was prepared according to the procedure described in Example 1, using 5 g of hyaluronic acid (Hyacare® 50 from Evonik) and 3.15 g of acrylic anhydride.

After purification, 3.2 g of a white solid were obtained.

Analyses:

¹H NMR D₂O: 0.74 OH unit functionalized for 4 OH units.

The hyaluronic acid obtained is 18%-functionalized with acrylate groups.

### EXAMPLE 4

### Hyaluronic Acid 12% Functionalized with Acrylic Anhydride

The polymer was prepared according to the procedure described in Example 1, using 5 g of hyaluronic acid (Hyacare® 50 from Evonik) and 1.58 g of acrylic anhydride.

After purification, 3.3 g of a white solid were obtained.

Analyses:

¹H NMR D₂O: 0.50 OH unit functionalized for 4 OH units.

The hyaluronic acid obtained is 12%-functionalized with acrylate groups.

### EXAMPLE 5:

Demonstration of the Tensioning Effect of the Polymers Used According to the Invention

The tensioning effect of the polymers of Examples 1 to 4 mixed with ethylenediamine was evaluated by means of an in vitro retraction test.

This test consists in comparing, in vitro, the tensioning capacity of the polymer+ethylenediamine mixture to be evaluated, relative to a reference tensioning polymer: Hybridur® 875 polymer dispersion from Air Products (aqueous dispersion at 40% by weight of particles of an interpenetrated network of polyurethane and acrylic polymers). The polymer to be evaluated is deposited on a nitrile rubber strip cut from a glove sold under the reference Safeskin Nitrile Criticial No. 038846 by the company Dominique Dutscher SA, having a surface area of 3.5 cm², stretched taut beforehand on a support. An aqueous solution containing the polymer to be evaluated is therefore deposited on the elastomer strip, by depositing 1.8 mg (of solids) of polymer.

26 μl of an aqueous solution containing 7% AM of Hybridur® 875 polymer are thus deposited on a nitrile rubber strip so as to thus obtain a reference tensioning strip, and 104 μl of an aqueous solution containing 3.5% AM of acrylate-functionalized hyaluronic acid to be evaluated, premixed with the aqueous solution of ethylenediamine according to the proportions specified hereinafter, are deposited on another strip.

After drying for 24 hours at ambient temperature (25° C.), the curving (retraction) of the strip treated with the grafted hyaluronic acid polymer mixed with the ethylenediamine is observed in comparison with that obtained with the control (Hybridur® 875).

The water resistance of the tensioning effect was then evaluated by immersing the rubber strips treated with the polymer to be evaluated in water at ambient temperature (25° C.) for 10 minutes.

Likewise, non-modified hyaluronic acid (Hyacare® 50 from Evonik) was evaluated using an aqueous solution at 3.5% AM, and also the grafted hyaluronic acid alone (polymer of Example 1) was evaluated using an aqueous solution at 3.5% AM.

An aqueous solution containing 1% by weight of ethylenediamine was prepared.

With the polymer of Example 1:

| | | | Amount of | | |
|---|---|---|---|---|---|
| | | | solution of | | |
| | | | acrylate- | | |
| | Proportion of | | grafted | | Volume of |
| | ethylenediamine | Amount of | hyaluronic | | the mixture |
| | (molar | ethylenediamine | acid of Ex 1 | Addition of | to be taken |
| Example | equivalent) | solution (μl) | (μl) | water (μl) | for a strip |
| 1a | 1 | 46 | 104 | / | 75 |
| 1b | 0.5 | 22 | 104 | / | 63 |
| 1c | 0.25 | 11.4 | 104 | 20 | 67 |
| 1d | 0.1 | 4.6 | 104 | 20 | 64 |

The following results were obtained:

| | | Tension effect after |
|---|---|---|
| Polymer tested | Tensioning effect | immersion in water |
| Hybridure 875 reference | correct | correct |
| Example 1a | greater than the | greater than the |
| | reference | reference |
| Example 1b | greater than the | greater than the |
| | reference | reference |
| Example 1c | greater than the | greater than the |
| | reference | reference |
| Example 1d | greater than the | greater than the |
| | reference | reference |
| Hyaluronic acid | Less than the | No tensioning effect |
| | reference | |
| Polymer Example 1 | Same as reference | Less than the |
| | | reference |

The results obtained show that the hyaluronic acid polymer of Example 1 mixed with ethylenediamine has a good tensioning effect, including after immersion in water. The tensioning effect obtained is greater than that of the non-modified hyaluronic acid and than that of the grafted hyaluronic acid alone.

With the polymer of Example 2:

| | Proportion of | | Amount of | |
|---|---|---|---|---|
| | ethylenediamine | | solution of | |
| | (in molar | Amount of | acrylate- | |
| | equivalent - | ethylene- | grafted | Volume of |
| | percentage relative | diamine | hyaluronic | the mixture |
| | to the acrylate | solution | acid of Ex 2 | to be taken |
| Example | functions available) | (μl) | (μl) | for a strip |
| 2a | 1 eq - 200% | 48 | 104 | 76 |
| 2b | 0.5 eq - 100% | 24 | 104 | 64 |
| 2c | 0.38 eq - 75% | 18 | 104 | 61 |
| 2d | 0.075 eq - 15% | 3.6 | 104 | 54 |

| | | Tensioning effect |
|---|---|---|
| | | after immersion in |
| Polymer tested | Tensioning effect | water |
| Hybridure 875 reference | correct | correct |
| Example 2a | equivalent to the | equivalent to the |
| | reference | reference |
| Example 2b | greater than the | greater than the |
| | reference | reference |
| Example 2c | greater than the | greater than the |
| | reference | reference |
| Example 2d | equivalent to the | equivalent to the |
| | reference | reference |
| Hyaluronic acid | Less than the | No tensioning effect |
| | reference | |

The results obtained show that the hyaluronic acid polymer of Example 2 mixed with ethylenediamine has a good tensioning effect, including after immersion in water. The tensioning effect obtained is greater than that of the non-modified hyaluronic acid.

With the polymer of Example 3:

| | Proportion of | | Amount of | |
|---|---|---|---|---|
| | ethylenediamine | | solution of | |
| | (in molar | Amount | acrylate- | |
| | equivalent - | of ethylene- | grafted | |
| | percentage relative | diamine | hyaluronic | Volume to |
| | to the acrylate | solution | acid of Ex 3 | be taken |
| Example | functions available) | (μl) | (μl) | for a strip |
| 3a | 0.36 - 200% | 20 | 104 | 62 |
| 3b | 0.18 - 100% | 10 | 104 | 57 |
| 3c | 0.13 - 75% | 7.2 | 104 | 55.6 |
| 3d | 0.03 - 15% | 1.7 | 104 | 52.8 |

| | | Tensioning effect |
|---|---|---|
| | | after immersion in |
| Polymer tested | Tensioning effect | water |
| Hybridure 875 reference | Correct | correct |
| Example 3a | equivalent to the | equivalent to the |
| | reference | reference |
| Example 3b | greater than the | greater than the |
| | reference | reference |
| Example 3c | greater than the | greater than the |
| | reference | reference |
| Example 3d | equivalent to the | equivalent to the |
| | reference | reference |
| Hyaluronic acid | Less than the | No tensioning effect |
| | reference | |

The results obtained show that the hyaluronic acid polymer of Example 3 mixed with ethylenediamine has a good tensioning effect, including after immersion in water. The tensioning effect obtained is greater than that of the non-modified hyaluronic acid.

With the polymer of Example 4:

| | Proportion of | | Amount of | |
|---|---|---|---|---|
| | ethylenediamine | | solution of | |
| | (in molar | Amount of | acrylate- | |
| | equivalent - | ethylene- | grafted | Volume of |
| | percentage relative | diamine | hyaluronic | the mixture |
| | to the acrylate | solution | acid of Ex 3 | to be taken |
| Example | functions available) | (μl) | (μl) | for a strip |
| 4a | 0.24 - 200% | 13.6 | 104 | 58.8 |
| 4b | 0.12 - 100% | 6.8 | 104 | 55.4 |
| 4c | 0.09 - 75% | 5.1 | 104 | 54.5 |
| 4d | 0.02 - 15% | 1.1 | 104 | 52.5 |

| | | Tensioning effect |
|---|---|---|
| | | after immersion in |
| Polymer tested | Tensioning effect | water |
| Hybridure 875 reference | correct | correct |
| Example 4a | equivalent to the | Less than the |
| | reference | reference |
| Example 4b | equivalent to the | Less than the |
| | reference | reference |
| Example 4c | equivalent to the | Less than the |
| | reference | reference |
| Example 4d | equivalent to the | Less than the |
| | reference | reference |
| Hyaluronic acid | Less than the | No tensioning effect |
| | reference | |

The results obtained show that the hyaluronic acid polymer of Example 4 mixed with ethylenediamine has a good tensioning effect, including after immersion in water. The tensioning effect obtained is greater than that of the non-modified hyaluronic acid.

### EXAMPLE 6:

An anti-wrinkle gel having the following composition was prepared:

| polymer of Example 1 | 3 | g |
|---|---|---|
| hydroxyethylcellulose | 0.5 | g |
| (NATROSOL ® 250 HHR CS from Ashland) | | |
| Preservatives | qs | |
| Water | qs 100 | g |

A similar composition was also prepared using the polymer of Example 2 or 3 or 4.

Just before the application to the skin, 405 mg of ethylenediamine are added to the gel.

The compositions obtained, as a mixture with ethylenediamine, when they are applied to the face, make it possible to effectively smooth out wrinkles.

1. Cosmetic process for caring for the skin comprising:
either the topical application to the skin of an extemporaneous mixture of a cosmetic composition comprising a hyaluronic acid polymer grafted with (meth)acrylate groups and an amine compound, or a cosmetic composition containing same;
or the sequential application to the skin of a cosmetic composition comprising a hyaluronic acid polymer grafted with (meth)acrylate groups and of an amine compound, or of a cosmetic composition containing same,
said amine compound having one or more primary amine and/or secondary amine groups.

2. Process according to claim 1, wherein the grafted hyaluronic acid polymer has a degree of grafting ranging from 10% to 80%.

3. Process according to claim 1 wherein the hyaluronic acid polymer is grafted with acrylate groups.

4. Process according to claim 1, wherein the grafted hyaluronic acid polymer has a weight-average molecular weight ranging from 5000 to 1 000 000 daltons.

5. Process according to claim 1, wherein the hyaluronic acid polymer is present in the composition in a content ranging from 0.1% to 10% by weight, relative to the total weight of the composition.

6. Process according to claim 1, wherein the amine compound comprises from 2 to 20 carbon atoms.

7. Process according to claim 1, wherein the amine compound is chosen from n-butylamine, tert-butylamine, isobutylamine, propylamine, n-hexylamine, glycine, ethanolamine, 3-aminopropanol, dopamine, 7-amino-4-methylcoumarin, 1,4-bis(3-aminopropyl)piperazine, 3-aminopropyltriethoxysilane (APTES), 3-aminophenylboronic acid, N-méthyl-1,3-diaminopropane, N-propyl-1,3-diaminopropane, N-isopropyl-1,3-diaminopropane, N-cyclohexyl-1,3-diaminopropane, 2-(3-aminopropylamino)ethanol, 3-(2-aminoethyl)aminopropylamine, bis(3-aminopropyl)amine, méthylbis(3-aminopropyl)amine, N-(3-aminopropyl)-1,4-diaminobutane, N,N-dimethyldipropylenetriamine, 1,2-bis(3-aminopropylamino)ethane, N,N′-bis(3-aminopropyl)-1,3-propanediamine, ethylenediamine, 1,3-propylenediamine, 1,4-butylenediamine, lysine, cystamine, xylènediamine, tris(2-aminoethyl)amine and spermidine.

8. Process according to claim 1, wherein the amine compound is chosen from amine-comprising polymers.

9. Process according to the claim 8, wherein the amine compound is an amine-comprising polymer chosen from poly((C₂-C₅)alkyleneimines), and in particular polyéthyleneimines and polypropyleneimines, especially poly(ethyleneimine)s; poly(allylamine); polyvinylamines and copolymers thereof, in particular with vinylamides; vinylamine/vinylformamide copolymers; polyamino acids which have NH₂ groups, such as polylysine; aminodextran; amino polyvinyl alcohol, acrylamidopropylamine-based copolymers; les chitosans;
polydimethylsiloxanes comprising primary amine groups at the chain end or on side chains, for example aminopropyl end or side groups, for instance those of formula (A) or (B) or (C): 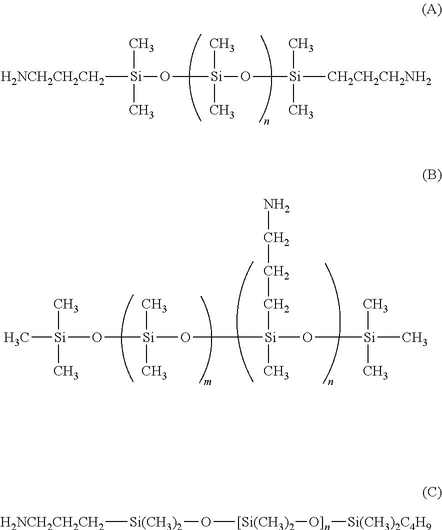
with:
in formula (A): the value of n is such that the weight-average molecular weight of the silicone is between 500 and 55 000;
in formula (B), the values of n and m are such that the weight-average molecular weight of the silicone is between 1000 and 55 000;
in formula (C), the value of n is such that the weight-average molecular weight of the silicone is between 500 and 3000;
amodimethicones of formula (D): 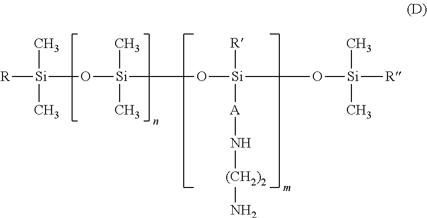
in which R, R′ and R″, which may be identical or different, each represent a C₁-C₄ alkyl or hydroxyl group, A represents a C₃ alkylene group and m and n are such that the weight-average molecular mass of the compound is between 5000 and 500 000 approximately;
polyether amines and in particular polyethylene glycols and/or polypropylene glycols comprising an amine function at the chain end (monoamine or diamine); polytetrahydrofurans (or polytetramethylene glycols) comprising an amine function at the chain end (monoamine or diamine), polybutadienes comprising an amine function at the chain end (monoamine or diamine);
dendrimers and polymers which are hyperbranched, comprising a primary or secondary amine function;
poly(meth)acrylates or poly(meth)acrylamides bearing primary or secondary amine side functions.

10. Process according to claim 1, wherein the amine compound is chosen from ethylenediamine, lysine and 3-aminopropyltriethoxysilane.

11. Process according to claim 1, wherein the amine compound is used according to an amine compound/grafted hyaluronic acid (meth)acrylate group mole ratio ranging from 0.1 to 10.

12. Process according to claim 1, wherein an extemporaneous mixture, prepared less than 5 minutes before the application to the skin, of the cosmetic composition comprising the (meth)acrylate-grafted hyaluronic acid polymer and of the amine composition, is applied to the skin.

13. Process according to claim 1, wherein the cosmetic composition comprising the (meth)acrylate-grafted hyaluronic acid polymer is first applied to the skin, then the amine compound or a cosmetic composition containing same is applied.

14. Process according to claim 1, wherein the amine compound, or a cosmetic composition containing same, is first applied to the skin, then the cosmetic composition comprising the (meth)acrylate-grafted hyaluronic acid polymer is applied.

15. Process according to claim 1, wherein the composition is in the form of an O/W emulsion or an aqueous gel.

16. Process according to claim 1, which is intended for attenuating wrinkles.

17. Cosmetic composition comprising as a skin tensioning agent a mixture of a hyaluronic acid polymer grafted with (meth)acrylate groups and an amine compound wherein said amine compound has one or more primary amine and/or secondary amine groups and comprises from 2 to 20 carbon atom.

18. Cosmetic composition comprising a mixture of a hyaluronic acid polymer grafted with (meth)acrylate groups and an amine compound wherein said amine compound has one or more primary amine and/or secondary amine groups.

19. Kit comprising a first cosmetic composition comprising a hyaluronic acid polymer grafted with (meth)acrylate groups and a second composition comprising an amine compound wherein said amine compound has one or more primary amine and/or secondary amine groups, the first and second compositions each being packaged in a distinct packaging assembly.

20. Process according to claim 2 wherein the hyaluronic acid polymer is grafted with acrylate groups.
